(19) United States
(12) Patent Application Publication
(10) Pub. No.: US 2003/0013163 A1
(43) Pub. Date: Jan. 16, 2003

(54) Method and device for producing shaped microbial cellulose for use as a biomaterial, especially for microsurgery

(76) Inventors: Dieter Klemm, Weimar (DE); Ulrike Udhardt, Golmsdorf (DE); Silvia Marsch, Stolzenberg (DE); Dieter Schumann, Jena (JP)

Correspondence Address: JORDAN AND HAMBURG LLP, 122 EAST 42ND STREET, SUITE 4000, NEW YORK, NY, 10168, US

(21) Appl. No.: 10/204,073

(22) PCT Filed: Feb. 13, 2001
(86) PCT No.: PCT/EP01/01621

(51) Int. Cl. C12P 19/04; C12N 1/21

(52) U.S. Cl. 435/101; 435/395; 435/252.3

(57) ABSTRACT

The use of exogenic materials for replacing blood vessels carries the risk of thrombosis and is therefore particularly unsuitable for microsurgical applications (inner vessel diameters of 1-3 mm and less), or only suitable under certain conditions. Replacements of blood vessels with a very small lumen in particular require biomaterials which guarantee that the surfaces of the prosthesis that come into contact with the blood are of a very high quality, and which reliably avoid this kind of thrombosis adhesion. The biomaterial is produced by immersing shaped body walls, especially of a glass matrix consisting of a glass tube and glass body, in a container of an inoculated nutrient solution so that the inoculated nutrient solution is drawn into the area between the walls of the shaped body and cultivation takes place in a moist, aerobic environment. In each subsequent cultivation process, an unused shaped body (glass body) is used as the shaped body wall for shaping the surface of the prosthesis material that is to come into contact with the blood when the biomaterial is used. This is the only sure way of reproducing the high surface quality of the vessel prosthesis and hereby reliably preventing thrombosis adhesion on the biomaterial used. The inventive method is particularly suitable for microsurgical applications, especially for replacing blood vessels and other internal hollow organs or as a cuff for covering nerve fibres, etc.

### BACKGROUND OF THE INVENTION

The invention relates to a method and device for producing shaped microbial cellulose for applications as biomaterial, in particular for microsurgical applications such as substitute for blood vessels and other internal hollow organs or as cuffs for enveloping nerve fibers or the like.

It is already known (for example, JP 3 165 774 A1) to use microbial cellulose as biomaterial in surgical applications, such as tissue implants, for example, for the abdominal wall, the skin, subcutaneous tissue, organs, for the digestive tract, for the esophagus, the trachea, and the urethra, as well as for cartilaginous tissue and for lipoplastics. Furthermore, it is known (for example, from JP 8 126 697 A2, EP 186 495 A2, JP 63 205 109 A1, JP 3 165 774 A1) that the microbial cellulose can be specifically shaped for its respective application in its production process, for example, in the shape of lamina, rods, cylinders and strips etc.

The following methods for manufacturing are described:

A plate is fixed at the surface of a culture solution which is inoculated with cellulose synthesizing microorganisms and the inculturing is executed. The result is a hollow cellulose cylinder, the cross-section of which corresponds to the surface of the liquid culture medium which is in contact to air.

Shaped microbial cellulose is synthesized on a gas permeable material (synthetic or natural polymers) in that the one side of the material is contacting a gas containing oxygen whereas the other one is contacting the liquid culture medium so that the microbial cellulose forms at the latter side and will subsequently be isolated.

Complex hollow fiber membranes will be obtained, for example, by coating porous surfaces (polymer compounds) with microbial cellulose in that the culture solution is given into the external (or internal) space of a separation membrane. Then air is directed through the external (or internal) space of the hollow fiber and a complex membrane is built up.

These methods involve the following disadvantages as to the quality of the inner surface of the built-up hollow body:

drying-out

formation of an inhomogeneous cellulose layer in the interior of the hollow cylinder which involves the danger that parts of the cellulose will be detached (cannot be applied for blood vessel substitutes, inparticular in the micro-range)

formation of complex products which not only consist of cellulose (affecting the bio-compatibility).

Furthermore, it is known (for example, from JP 3 272 772 A2) to use shaped bio-material as micro-lumenal blood vessel substitutes, whereby the vessel prosthesis is cultivated on a hollow support which is permeable to oxygen (for example cellophane, Teflon, silicon, ceramic material, non-woven texture, fibers).

It is disadvantageous that the hollow cylinders produced in this way do not have a sufficiently smooth inner surface so that clots can deposit in the inserted blood vessel prosthesis. The surface quality of these inner surfaces is the more significant, the smaller is the diameter of the vessel substitute, since vessels of narrow lumen are particularly susceptible to occlusions by clot depositions. The use of these prostheses in microsurgery, when vessel diameters of 1-3 mm or less are concerned, is therefore extremely problematic, or even impossible.

In EP 396 344 A3 there are described a hollow cellulose, produced by a microorganism, a process for producing said cellulose, as well as an artificial blood vessel formed of said cellulose.

The first process for producing the hollow microbial cellulose comprises the inculturing of a cellulose synthesizing microorganism on the inner and/or outer surface of a hollow support permeable to oxygen, said support being made of cellophane, Teflon, silicon, ceramic material, or of a non-woven and woven material, respectively. Said hollow support permeable to oxygen is inserted into a culture solution. A cellulose synthesizing microorganism and a culture medium are added to the inner side and/or to the outer side of the hollow support. The inculturing takes place under addition of an oxygenous gas (or liquid) also to said inner side and/or to the outer side of the hollow support. A gelatinous cellulose of a thickness of 0.01 to 20 mm forms on the surface of the hollow support. Due to the interaction of the cellulose synthesizing microorganism with the produced cellulose and the hollow support, a composite of cellulose and a hollow support results. Provided that the cellulose is not bound to the support, the latter will be removed after the synthesis of the cellulose and a hollow shaped article will be obtained which exclusively consists of cellulose. The cellulose produced in this way will be cleaned from the cells of the microorganism or from components of the culture solution by means of dilute alkali, dilute acid, an organic solvent and hot water, alone or in a combination thereof.

The disadvantage of this method again results from the formation of an inhomogeneous cellulose layer in the interior of the hollow cylinder involving the danger that parts of the cellulose will detach (which is problematic for blood vessels, particularly in the micro range).

As a second process for formation of a hollow microbial cellulose the impregnation, an after-treatment, if necessary, and a cutting of the cellulose generated by the microorganism is described in EP 396 344 A3. A vessel filled with culture solution is inoculated with the microorganism. The microbial cellulose which has formed is impregnated with a medium and, if necessary, after-treated, frozen or compacted. Thus, the liquid component will be retained between the fibers, which form the microbial cellulose, in order to prevent free movement of the liquid component. Then the cutting procedure is carried out. As medium can be used, alone or in mixtures: polyols such as glycerol, erythrol, glycol, sorbitol, and maltitol, saccharides such as glucose, galactose, mannose, maltose, and lactose, natural and synthetic polymeric substances such as polyvinyl alcohol, polyvinyl pyrrolidone, polyethylene glycol, carboxymethylcellulose, agar, starch, alginate, xanthan gum, polysaccharides, oligosaccharides, collagen, gelatin, and proteins, as well as polar solvents soluble in water such as acetonitrile, dioxane, acetic acid, and propionic acid.

This method includes the following disadvantages with respect to the manufacturing expenditures and to the quality of the inner surface of the formed hollow body:

no direct formation during the biosynthesis

hydrophilic properties of the microbial cellulose, which, for example, determine the roughness of the inner surface as well as the biocompatibility, are changed.

The third process for producing the hollow microbial cellulose, the manufacturing by way of two glass tubes of different diameter is described in EP 396 344 A3. The glass tubes are inserted into one another and the inculturing of the microorganisms is carried out in the space between the two tube walls within 30 days. The result is microbial cellulose of a hollow cylindrical shape which due to its good compatibility to the living organism, specially to blood, can be used as a blood vessel substitute in the living body. The blood compatibility (antithrombogenic property) was evaluated by the blood vessel substitute test under use of a grown-up half-breed dog. Parts of the descending aorta and of the jugular vein of the dog were replaced by the artificial blood vessel having an inner diameter of 2-3 mm. After one month the artificial blood vessel was removed and examined as to the state of the adhesion of clots. There was a slight deposition of clots in the range of the suture and a non-insignificant adhesion of clots was observed over the entire inner surface of the artificial blood vessel (refer to example 10 of the specification). There is provided a biologically comparatively well compatible hollow cylindrical cellulose which in particular can serve as a blood vessel substitute of a diameter of smaller than 6 mm. However, due to the danger of deposition of clots, an application in vessels of small lumen (2-3 mm in the example described) cannot be considered as harmless. Moreover, microsurgical applications require still smaller vessel diameters of 1 mm and below. Here the application of these vessel prostheses seems to be impossible due to the mentioned adhesion of clots upon the inner wall.

### SUMMARY OF THE INVENTION

Therefore, it is an object of the present invention to provide a method for producing shaped biomaterial, in particular for microsurgical applications as blood vessel substitutes of 1-3 mm diameter and smaller which ensures a very high and reproducible quality of the prosthesis material surfaces contacting the blood and which reliably avoids a clot adhesion on said surfaces.

The biomaterials have to be tissue compatible and blood compatible, and they have also to permit production at the lowest possible manufacturing expenditures including the manufacturing time, also in any desired shape and also in variable hollow cylindrical designs.

The culture medium is rendered sterile in known manner, inoculated with cellulose generating bacteria, for example, with a strain of the microorganism Acetobacter xylinum generating a form-stable cellulose layer, and then cultivated in a space between the walls of a shaping body at a temperature of, for example, between 28° C. and 30° C. The biomaterial (cellulose) resulting by the inculturing is isolated from the walls of shaping body as well as subjected to a cleaning procedure (refer to EP 369 344 A3).

The inoculated culture medium is not filled into the space in-between the walls of the shaping body, for example, of a glass matrix preferably consisting of glass bodies detachable from each other, but according to the invention, during inculturing the walls of the shaping body (glass matrix) are immersed into a vessel containing the inoculated culture medium so that the culture medium is drawn-in into the space between the walls of the shaping body by capillarity. In this way and throughout the entire inculturing procedure a moist aerobic environment is ensured in the vessel for cellulose formation.

For producing hollow cylindrical cellulose as a blood vessel substitute, a glass matrix, known per se, comprised of an outer glass tube and a glass body fixedly arranged in axial symmetry relative to and in said glass tube, is immersed into the inoculated culture medium which is in said vessel, for example, an Erlenmeyer flask. After inculturing the glass matrix is removed from the vessel and disassembled for taking out the produced cellulose.

In each inculturing process a respective unused shaping body of high surface quality is used as a shaping body wall for shaping the prosthesis material surface which comes into contact with the blood when the biomaterial is applied. Thus even microscopically small deposits of culture medium particles and cellulose fibers, if any, are reliably prevented from depositing on the shaping body wall which otherwise, in the case of a reuse of the shaping body, in spite of even the most thorough cleaning might affect a change of the adhesion conditions on the shaping body wall for the growing cellulose. This means with respect to the cylindrical glass matrix that for each new inculturing process an unused glass body for shaping the inner wall of the vessel substitute to be produced has to be fixed in the outer glass tube. The cylindrical glass body can advantageously be selected from commercially available standard measure melting-point capillaries.

By these method steps it was surprisingly found that there was not encountered, in a period of time corresponding to that described in the example of EP 396 344 A3, any comparable deposit of clots. The surface quality of the prosthesis material surfaces which are produced in this manner and which contact the blood when implanted is reproducibly very high and the danger of a clot adhesion is very low. Thus, the biomaterials produced according to the invention are very well suited as permanent blood vessel substitutes in microsurgical applications, in particular for vessel diameters of 1-3 mm and smaller.

Further advantages of the proposed method are the short inculturing times (already after 7 to 14 days a cellulose layer of stable shape has formed in the glass matrix) as well as the good distribution of the inoculation culture in the medium by virtue of the inoculation of the liquid culture medium with a liquid parent culture (“liquid-liquid inoculation”).

The tubular biomaterial produced by means of a cylindrical glass matrix can be used with advantage not only as vessel prostheses, but also as cuffs for enveloping nerve fibers and the like, as well as for exercising material, in particular for training microsurgical techniques. The number of experimental animals can be reduced by the last-mentioned application. The exercising material used up to now consists, for example, of gum and can only incompletely simulate operation conditions which should be as real as possible.

The independent claims set out further advantageous embodiments of the invention.

Furthermore, a useful device for carrying out the production method is disclosed. In this device the inner glass cylinder of the glass matrix which is renewed for each inculturing process is fixed, readily detachable and in stable position, in the outer glass tube to the ends of the cylinder by way of sleeve-like elastic rings. In this way the glass matrix can be disassembled at the lowest possible expenditures for time and handling, whereby the outer glass tube can be reused and the inner glass cylinder can be exchanged as mentioned above. Furthermore, the produced hollow cylindrical cellulose can be isolated, material-preserving and surface-preserving, without any problems. The circulation of the culture medium and of the air to the interspace of the glass matrix and from the same, respectively, is ensured by openings of the glass tube which are arranged in the range between the elastic rings of the glass matrix. The use of such a device is efficient since only the inner cylindrical glass body has to exchanged in the subsequent inculturing process and since cumbersome cleaning steps can be omitted or are reduced to a minimum.

In order to increase the output of the biomaterial to be produced a plurality of glass matrices can be simultaneously immersed for said inculturing into the vessel containing the inoculated culture medium.

The manufacturing method is not restricted to the hollow cylindrical shaping of the biomaterial and not to microsurgical applications.

### DETAILED DESCRIPTION OF THE INVENTION

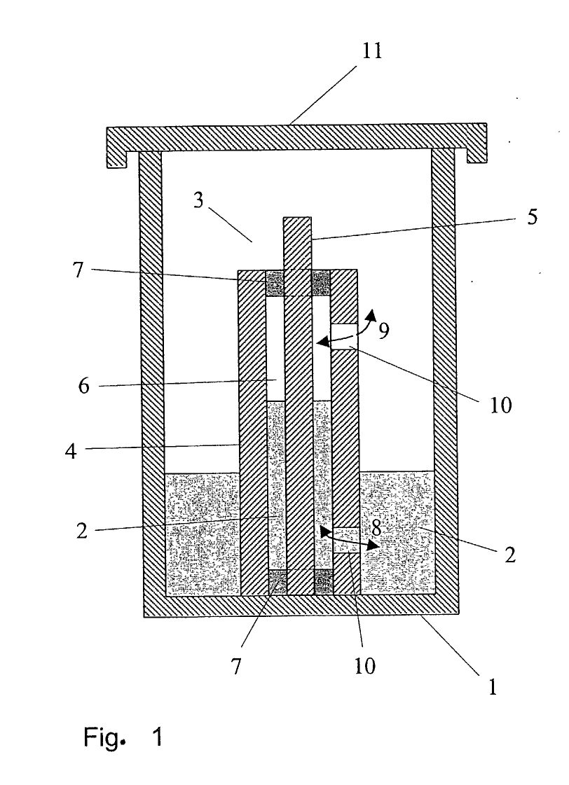

The invention will be explained hereinafter in more detail by virtue of one embodiment under reference to FIG. 1.

A vessel 1 of a capacity of 50 ml was filled with 20 ml of a culture medium 2 (Schramm-Hestrin-medium) which contains, per liter distilled water, 20.00 g of glucose free of water, 5.00 g of bactopeptone, 5.00 g of yeast extract, 3.40 g of disodium-hydrogenphosphate dihydrate, and 1.15 g of citric acid monohydrate and which exhibits a pH value between 6.0 and 6.3. The culture medium 2 was steam sterilized at 120° C. for 20 minutes and than inoculated with the bacterium Acetobacter xylinum (AX 5, strain collection of the Institute of Biotechnology Leipzig) from a 10 days old liquid strain culture (Schramm-Hestrin-medium). Thereafter, a sterilized glass matrix 3, constituted of an outer glass tube 4 and an inner glass body 5 of a cylinder diameter of 0.8 mm fixed in axial symmetry within and relative to said glass tube 4, is immersed into the vessel 1. Due to the capillary effect, a space 6 between the outer glass tube 4 and the inner glass body 5 fills with the inoculated culture medium 2 of the vessel 1. The cultivation time was 14 days at a temperature between 28° C. and 30° C. During this cultivation period a white microbial cellulose formed in both, the vessel 1 and in the space 6 of the glass matrix 3.

The glass matrix 3 was removed from the vessel 1 and disassembled, the cylindrical microbial cellulose which has formed in the space 6 of the glass matrix 3 was isolated, washed thoroughly with water, treated for 10 minutes with boiling aqueous 0.1 N caustic soda solution and then again washed thoroughly with water in order to obtain a microvessel prosthesis of an inner diameter of 0.8 mm, a wall thickness of 0.7 mm an a length of up to 1 cm.

The blood compatibility of this microvessel prosthesis was evaluated by an animal experimental study, in which parts of the carotis of WISTAR-rats were replaced by the produced artificial blood vessel. To this end and before the operation, the water contained in the swollen cellulose material was exchanged for physiological saline solution. Right after the operation an unobstructed blood flow could be observed.

After one month the artificial blood vessel was removed which, by embedding into the connective tissue and the formation of small blood vessels within the connective tissue, had been very well integrated into the animal body and was completely patent. The state of the artificial prosthesis, the anastomoses ranges and the part of the carotis distally to the second anastomosis with the artificial blood vessel was examined histologically and by electron microscope. There was no thrombogenesis and no proliferation process found, neither in the suture ranges, nor in the bridging graft, nor in the blood vessel. The inner surface of the prosthesis including the anastomosis range was “biologized”, that is, completely covered with endothelial cells (formation of a neo-intima). The inner surface of the anastomoses was flat and completely unobstructive. These results were confirmed by a total of 20 animal experiments.

For a repeated use of the glass matrix 3 in a subsequent cultivation procedure, the glass body 5 was substituted for an unused glass body 5 and the described process was carried out again.

The glass body 5 is fixed by sleeve-like silicon rings within the glass tube 4 in order to fix the glass body 5 in a stable position within the glass tube 4 at the lowest possible manipulation expenditures and to permit a dismounting of the glass matrix 3 at even the same lowest possible expenditures and, above all, material preserving with respect to the produced cellulose. However, to ensure a culture medium exchange 8 and a substantially unobstructed air circulation 9 the glass tube 4 is provided with openings 10 in the range between the silicon rings 7. The vessel 1 is closed by a cover 11 during the cultivation process to ensure sterility and a moist and aerobic environment within the vessel 1.

### LIST OF REFERENCE NUMERALS

| | |
|---|---|
| 1 | vessel |
| 2 | culture medium |
| 3 | glass matrix |
| 4 | glass tube |
| 5 | glass body |
| 6 | (inter-) space |
| 7 | silicon ring |
| 8 | culture medium exchange |
| 9 | air circulation |
| 10 | opening |
| 11 | cover |

1. Method for producing shaped microbial cellulose for application as biomaterial, in particular for microsurgical applications, in which a sterilized culture medium is inoculated with cellulose generating bacteria, for example, with a strain of the microorganism Acetobacter xylinum generating a form-stable cellulose layer, and the bacteria are cultivated in a space between the walls of a shaping body and in which the biomaterial (cellulose) resulting from the cultivation is isolated from the walls of the shaping body as well as subjected to a cleaning procedure, characterized in that the walls of the shaping body are immersed into a vessel containing the inoculated culture medium and the microorganism is cultivated for cellulose formation in both, in the vessel and in the space between the walls of the shaping body in a moist and aerobic environment, and in that in each inculturing process an unused shaping body of high surface quality is used as a shaping body wall for shaping the prosthesis material surface which, when the biomaterial is applied, comes into contact with the blood.

2. Method as claimed in claim 1, characterized in that a glass matrix of glass bodies being preferably detachable from each other is used for the shaping body walls between which the microorganism is cultivated.

3. Method as claimed in claim 2, characterized in that for producing hollow cylindrical biomaterial, a glass matrix, comprised of an outer glass tube and a glass body inserted into said glass tube in axial symmetry to and being of smaller diameter than the latter, is inserted into the vessel containing the inoculated culture medium.

4. Method as claimed in claim 2, characterized in that for simultaneously producing a plurality of biomaterials, a plurality of glass matrices is inserted into the vessel containing the inoculated culture medium.

5. A device for carrying out the method as claimed in claim 3, characterized in that at least one glass matrix (3), comprised of an outer glass tube (4) and a glass body (5) inserted into said glass tube (4) in axial symmetry to and being of smaller diameter than the latter, is immersed into a vessel (1) containing the inoculated culture medium (2), whereby the inner glass body (5), for the purpose of an easy manipulation and a position stable and easily detachable centering within said outer glass tube (4) in axial symmetry to the latter, is fixed by way of elastic rings (7), preferably made of silicon, under provision of a culture medium exchange (8) and an air circulation (9) into, respectively, from out of an interspace (6) of the glass matrix (3), said interspace being for shaping said biomaterial to be produced.

6. Device as claimed in claim 5, characterized in that the culture medium exchange (8) and the air circulation (9) is ensured by at least one respective opening (10) of the outer glass tube (4) within the range of the glass matrix (3) between the elastic rings (7).

7. Device as claimed in claim 5, characterized in that an Erlenrmeyer flask, known per se, is used as the vessel (1) into which the glass matrix (3) is immersed.
